(19) United States
(12) Patent Application Publication
(10) Pub. No.: US 2017/0100925 A1
(43) Pub. Date: Apr. 13, 2017

(54) METHOD OF MAKING A PRINTED ELASTIC LAMINATE

(71) Applicant: MONDI Gronau GmbH, Gronau (DE)

(72) Inventors: Dieter HOMOELLE, Ochtrup (DE); Marcus SCHOENBECK, Versmold (DE)

(21) Appl. No.: 15/385,090

(22) Filed: Dec. 20, 2016

Related U.S. Application Data
(62) Division of application No. 14/268,342, filed on May 2, 2014.

(30) Foreign Application Priority Data
May 3, 2013 (EP) 13 166 524.2

(51) Int. Cl. B32B 38/00; A61F 13/15; B32B 37/20

(52) U.S. Cl. CPC B32B 38/145; B32B 37/203; B32B 38/1875; A61F 13/15699; A61F 13/15739; B32B 2307/51; B32B 2555/00; B32B 2555/02; A61F 2013/15715

(57) ABSTRACT

A method of making a printed, elastic laminate has the steps of transversely stretching and then relaxing a longitudinally elongated elastic film then printing a motif on the film. The printed elastic film is cut into longitudinally extending strips that are then bonded transversely next to each other between two surface webs through at least one of which the motif is visible to form a laminate. The laminate is then transversely stretched at least at the strips.

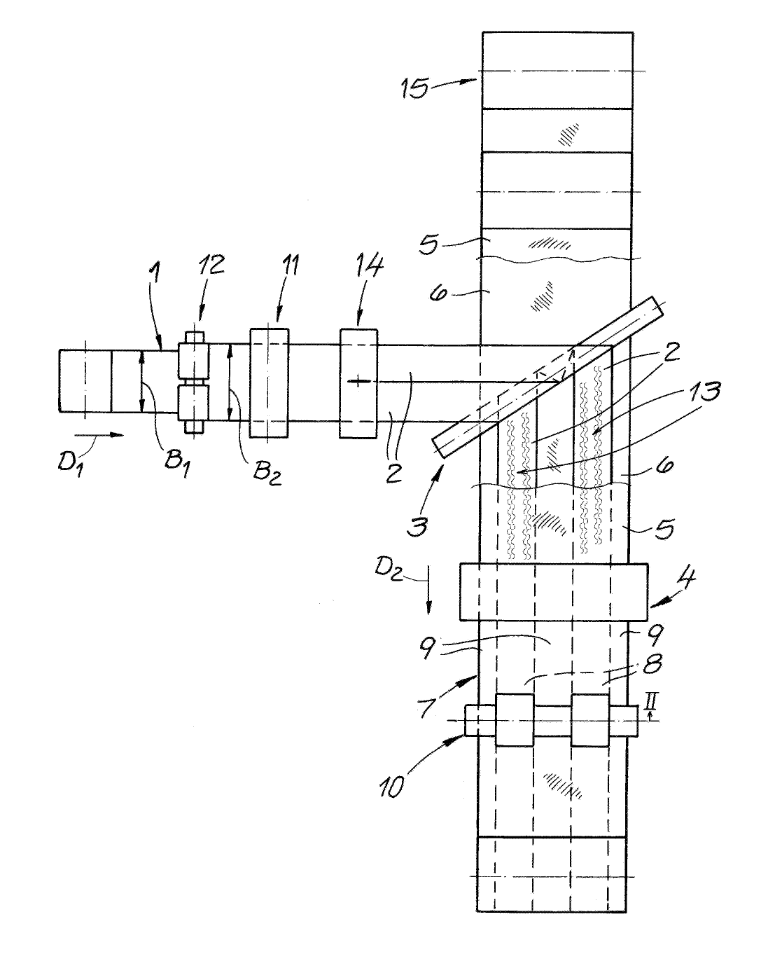

### CROSS REFERENCE TO RELATED APPLICATION

This application is a division of copending patent application Ser. No. 14/268,342 filed 2 May 2014 with a claim to the priority of EP 13 166 524.2 filed 3 May 2013.

### FIELD OF THE INVENTION

The present invention relates to an elastic laminate. More particularly this invention concerns method of making a printed elastic laminate.

### BACKGROUND OF THE INVENTION

An elastic laminate is typically made by cutting an elastic film into strips that are laminated next to each other between two textile surface webs to form a laminate that is stretched transversely in areas rendered elastic by the strips relative to the web. The transverse stretching, which is also referred to as mechanical activation, improves the elastic properties of the laminate transversely of the direction of the material web (CD direction).

The textile surface webs can be made of a nonwoven, woven, or a knitted material. The strips can be configured in a single layer or a plurality of layers containing a sufficient quantity of an elastomeric polymer. The strips are glued or welded to the surface webs. One can die-cut elastic elements for hygiene products from the laminate, particularly elastic closure strips for diapers having an elastic middle region and adjacent thereto less elastic ends. The ends that are not elastic or less elastic are used for fastening hook-and-loop closure elements such as, for example, hook-fastener tapes thereto, and for attaching the elastic element to the inelastic regions of a diaper. The laminate is manufactured as a broad web that includes a plurality of laminated elastic strips. It is then possible to die-cut from the resulting multipurpose material the closure strips that are needed to make diapers. Such a method is known, for example, from U.S. Pat. No. 7,470,340.

U.S. Pat. No. 8,529,725 discloses a method of making a printed diaper manufactured from elastic closure elements and inelastic diaper sections. The elastic closure elements as well as the inelastic diaper sections are made of laminates that include a textile surface web and a film liner or film carrier. The elastic closure elements as well as the inelastic part of the diaper carry an imprint whose printed images are harmonized at the edges of elastic and inelastic regions such that there results a uniform and attractive overall image.

U.S. Pat. No. 7,896,858 also discloses diapers having printed elastic and inelastic sections where the printed images are able to complement each other to form a uniform motif.

However, the job of printing the image onto the laminate using the method described above has not been satisfactorily resolved to date. If a previously printed textile surface web is used for making the laminate, there exists the problem that the printed image previously applied to the textile surface is destroyed at least in part by tears and shifting of the fibers during the mechanical activation of the laminate and any later use by the consumer. Moreover, problems can result that are related to the positioning of the printed image that is a surface web and the elastic strips that are added thereto. To be considered therein is the fact that different web tensions are transferred to the strips during cutting of the film into strips, advance of the web of elastic strips, and also during laminating; this can result in a change of strip width. If the printed image is made of a random printed motif having elements that are distributed across the entire print width, alignment errors of the elastic strips and the printed image are often not noticeable. However, if the regions made elastic by the strips are printed with, for example, a striped motif, optical inaccuracies are clear between the position of the strip and the printed motif if elastic elements manufactured from the laminate are extensively stretched during use, for example, when closing a diaper.

### OBJECTS OF THE INVENTION

It is therefore an object of the present invention to provide an improved method of making a printed elastic laminate.

Another object is the provision of such an improved method of making a printed elastic laminate that overcomes the above-given disadvantages, in particular that has an improved appearance of the printed image.

### SUMMARY OF THE INVENTION

A method of making a printed, elastic laminate has according to the invention the steps of printing a motif on a longitudinally elongated elastic film. The printed elastic film is cut into longitudinally extending strips that are then bonded transversely next to each other between two surface webs through at least one of which the motif is visible to form a laminate. The laminate is then transversely stretched at least at the strips.

Due to the fact that the elastic film is provided with the imprint, even while using the laminate that has been printed, correct alignment of the printed motif relative to the elastic region of the laminate is always ensured. This gives the advantage that, when stretching the elastic strip, the printed image is evenly and reversibly stretched along with it. Furthermore, the printed motif is visible from the front side as well as from the back side of the laminate, for example, through a nonwoven textile surface web, such that the laminate is optically equally attractive from the front as well as from the back. For example, the elastic film can be printed with a striped motif consisting of parallel, colored stripes extending in the direction of the web of the elastic film.

Known continuous printing methods can be used for printing the elastic film. Rotary printing processes are preferred that allow for printing the elastic film at high web speeds. The goal is web speeds of approximately 400 m/min. Gravure printing and flexography methods are advantageous processes, flexography being particularly preferred because it is possible to use one central cylinder for a plurality of color systems. Digital printing that transfers the printed image directly from a computer into a printing machine without the use of a static medium are not excluded. In particular, ink-jet printing methods are conceivable that generate a printed image by deflecting small ink drops.

According to a preferred embodiment of the invention, the elastic film is stretched transversely of the direction of the web before the printing process, then printed after elastic relaxation, and subsequently cut into strips. The stretching of the elastic film constitutes a mechanical preactivation of a layer of the laminate and results in improved stretching behavior of the laminate. The preactivation of the elastic film has a positive effect on the course of the expansion force and provides easy stretching action of the laminate over a large area and at an expansion limit that is determined by the preactivation of the elastic film and after which the expansion resistance increases strongly. The return behavior of the laminate after tension is removed can also be improved if the elastic film is preactivated by transverse stretching before laminating it into the laminate. Any preactivation of the elastic film cannot replace but can only supplement the mechanical activation of the laminate. Even when the elastic film is preactivated, it is still necessary for the laminate to be stretched transversely of the direction of the web in regions that are to be rendered elastic by laminated strips.

A preferred embodiment of the method according to the invention provides that the elastic film is stretched transversely of the web by more than 50% and has a width after reverse expansion that is greater than the starting width of the elastic film by 10% to 30% before it was stretched. The term “stretching” is used despite the fact that the expansion is not completely reversible but that some plastic deformation results in the film having a larger width following the reverse expansion. Later activation of the laminate essentially affects the structure of the textile surface webs. The transverse stretching of the preactivated elastic film, on the other hand, is for the most part reversible. The printed image that is applied to the preactivated elastic film thus does not undergo any further disadvantageous changes during subsequent activation of the laminate. Correspondingly, it is possible to improve the quality of the printed image on the elastic laminate if the elastic film is only printed following preactivation, during which the elastic film is expanded transversely and then released.

It is possible to use a stretch-rolling apparatus of profile rollers that mesh with each other for the stretching action of the elastic film and/or the laminate.

Preferably a polyolefin elastomer film is used as the elastic film. When using an polyolefin-elastomer-based elastic film, preactivation of the elastic film is especially advantageous.

In addition, it is also possible to use as an elastic film a single-layer or multilayer film having an elastomeric core layer made a material of styrene-isoprene-styrene block copolymers (SIS), styrene-butadiene-styrene block copolymers (SBS), styrene-ethylene-butylene-styrene block copolymers (SEBS), polyurethanes, ethylene copolymers, or polyether block amides.

After preactivation and printing, the elastic film is cut into strips. The strips are guided across a deflector and can be supplied as parallel strips to a laminator where the strips are laminated between the textile surface webs. The elastic strips are position at a transverse spacing from each other. The transverse spacing between the strips can be adjusted by the position of the deflector. In the gaps between the elastic strips, the surface webs are directly bonded to each other. It is within the scope of the invention to use reinforcement strips that are laminated between the elastic strips so as to reinforce the gaps between the elastic strips. It is thus possible to constitute elastic and inelastic regions inside the laminate.

### BRIEF DESCRIPTION OF THE DRAWING

The above and other objects, features, and advantages will become more readily apparent from the following description, reference being made to the accompanying drawing whose sole figure is a schematic top view of an apparatus for carrying out the method of this invention.

### SPECIFIC DESCRIPTION OF THE INVENTION

As seen in the drawing, an elastic film 1 moving in a first travel direction D₁ is cut at 14 into strips 2 that are guided across a deflector 3 and supplied in a second direction D₂ perpendicular to the direction D₁ to a laminator 4 as parallel strips 2. The strips 2 are laminated between textile surface webs 5 and 6 that are pulled off a supply 15 then fed from above and below to the strips 2. The strips 2 and the textile surface webs 5 and 6 are glued together or connected to each other thermally in the laminator 4. The view shows that the elastic strips 2 are laminated in longitudinally extending regions 8 at a spacing from each other between the surface webs 5 and 6 and that the textile surface webs 5 and 6 are directly connected to each other in gaps 9 between the elastic strips 2 and in edge regions 9 flanking the regions 8. This way, elastic regions 8 as well as inelastic regions 9 are created in the laminate 7.

The laminate is then supplied to an activator 10 in which the laminate 7 is stretched transversely at the regions 8 rendered elastic by the laminated strips 2 relative to the travel direction D₂ of the web. As disclosed in copending application attorney's docket 30677 (incorporated herein by reference), a stretch-roller apparatus having profile rollers that mesh with each other is used for stretching the laminate 7. Stretching modifies the textile structures of the surface webs, and the expansion property of the laminate 7 is improved transversely of the longitudinal web direction that here is also the travel direction D₂. Following activation, the laminate is easily expandable in the CD direction by minimal force to an expansion limit that is determined by the activation.

The textile surface webs 5 and 6 are made of, in particular, nonwoven fabric; woven or knitted fabrics are also possible. A single-layer or multilayer elastomer film can be used as elastic film 1 having an elastomer core layer made of styrene-isoprene-styrene block copolymers, styrene-butadiene-styrene block copolymers, styrene-ethylene-butylene-styrene block copolymers, polyurethanes, ethylene copolymers, or polyether block amides. An elastic blown film made of a polyolefin elastomer is preferred.

Before cutting the film into strips 2, the elastic film 1 is printed in a printing station 11 with a motif shown only at 13 downstream of the deflector 3. This motif 13 is visible through the textile surface webs 5 and 6 of the laminate 7. Printing is preferably done by a rotary printing method, particularly flexography. The printed motif can be, for example, a striped motif made of parallel colored stripes that extend in the longitudinal direction of the elastic film of the web.

Before printing, the elastic film 1 is stretched at a station 12 transversely of the web by more than 50%. Preferably, an expansion by 100% to 500% is effected relative to a starting width of the elastic film. After the elastic relaxation, the elastic film 1 has a width B₂ that is larger by 10% to 30% than a starting width B₁ of the elastic film. Following the relaxation, the elastic film 1 is printed and subsequently cut into strips 2.

Expanding and/or stretching the elastic film 1 in the transverse-stretching station 12 constitutes preactivation, which has considerable advantages with regard to the expansion values of the laminate 7. Due to preactivation of the elastic film 1 prior to the printing process, it is also possible to improve the printed image of the elastic laminate 7, the reason for this being that during stretching of the laminate 7, the printed image is evenly and reversibly expanded along with the laminate, and preactivation of the elastic film 1 results in the laminate 7 completely resetting itself following stretching in the activation apparatus 10.

We claim:

1. A method of making a printed, elastic laminate, the method comprising the steps of:
transversely stretching a longitudinally elongated elastic film;
transversely elastically relaxing the transversely stretched film;
printing a motif on the relaxed film;
cutting the printed elastic film into longitudinally extending strips;
bonding the strips transversely next to each other between two surface webs through at least one of which the motif is visible to form a laminate; and
transversely stretching the laminate at least at the strips.

2. The method defined in claim 1, wherein the motif is formed as a pair of parallel longitudinally extending colored stripes.

3. The method defined in claim 1, wherein the printing is done printed by flexography.

4. The method defined in claim 1, wherein the printing is dome by gravure or digital printing.

5. The method defined in claim 1, wherein stretching transversely stretches the elastic film by more than 50% to have a width after elastic relaxation that is larger by 10% to 30% than a width of the elastic film prior to stretching.

7. The method defined in claim 1, wherein the stretching is effected by profile rollers that mesh with each other.

8. The method defined in claim 1, wherein the elastic film is a polyolefin elastomer.

9. The method defined in claim 1, wherein the elastic film has an elastomer core layer made from styrene-isoprene-styrene block copolymers, styrene-ethylene-butylene-styrene-block copolymers, polyurethanes, ethylene-copolymers or polyether block amides.

10. The method defined in claim 1, further comprising the step before laminating of:
guiding the strips over a deflector and thereafter sandwiching the strips between the surface webs before laminating the surface webs and strips to form the laminate.

11. The method defined in claim 1, wherein the strips are spaced transversely from each other by a longitudinally extending gap when they are laminated between the surface webs, to form longitudinally extending and spaced elastic and inelastic regions in the laminate.
